(19) United States
(12) Patent Application Publication
(10) Pub. No.: US 2015/0133897 A1
(43) Pub. Date: May 14, 2015

(54) LIPOSUCTION ABSORPTION CARTRIDGE

(71) Applicant: Constantino G. Mendieta, Miami, FL (US)

(72) Inventor: Constantino G. Mendieta, Miami, FL (US)

(21) Appl. No.: 14/539,825

(22) Filed: Nov. 12, 2014

Related U.S. Application Data
(60) Provisional application No. 61/903,367, filed on Nov. 12, 2013.

(51) Int. Cl. A61M 1/00

(52) U.S. Cl. CPC A61M 1/0058; A61M 1/0094
USPC 604/542

(57) ABSTRACT

An absorption cartridge for use in removing liquids from a liposuction collection flask may have a hollow body containing absorbing materials and/or a desiccant. The absorbing materials or desiccants may be incorporated into a hollow body or supported about a frame. The cartridge may be inserted into the collection flask by means of a slot in the lid, or may be placed entirely within the collection flask. The cartridge may be capable of withstanding known sterilization techniques.

### CROSS-REFERENCE TO RELATED APPLICATIONS

This application claims priority to U.S. Provisional Application Ser. No. 61/903,367 filed on Nov. 12, 2013, the contents of which are hereby incorporated in their entirety.

### STATEMENT REGARDING FEDERALLY SPONSORED RESEARCH OR DEVELOPMENT

Not Applicable.

### NAMES OF PARTIES TO A JOINT RESEARCH AGREEMENT

Not Applicable

### REFERENCE TO SEQUENCE LISTING, A TABLE, OR A COMPUTER PROGRAM LISTING APPENDIX SUBMITTED ON A COMPACT DISC AND INCORPORATION-BY-REFERENCE OF THE MATERIAL

Not Applicable.

### COPYRIGHT NOTICE

Not Applicable

### BACKGROUND OF THE INVENTION

1. Field of the Invention

The present invention relates to systems and methods for absorbing liquids within a flask. More particularly, the invention relates to systems and methods for removing excess liquids from lipoaspirate in a collection flask in a sterile manner.

2. Background Information

The process of liposuction, or the harvest of subcutaneous fat, is commonly performed by introducing a cannula into a desired location through a small incision. A lipoaspirator machine is used to apply suction through the cannula which then removes adipose (fat) tissue. This liposuction method, also called lipoaspiration, uses this vacuum pressure to draw fat tissue into a collection flask connected in series between the suction cannula and a vacuum pump. The material deposited into the collection flask is generally referred to as lipoaspirate.

Several techniques have been developed to improve the liposuction process. A jet of water may applied to adipose tissue to loosen it from surrounding tissue. Some liposuction techniques use a relatively high pressure vacuum to more easily remove the fat tissue. Other techniques utilize ultrasound to lyse the adipocytes, making them less cohesive and easier to remove via suction. These techniques damage the adipose tissue, resulting in a lipoaspirate composed mainly of damaged or disrupted adipocytes which are discarded.

In the past several years, new uses have been found for healthy, intact adipocytes recovered from a lipoaspirate. Adipose tissue has been found to include stem cells that may be used in a to grow new cells of all types. Healthy adipose tissue isolated from lipoaspirate may also be used in other cosmetic surgery procedures, being reinserted into a patient at a desired location. The use of a patient's own adipose tissue rather than other materials when sculpting breast or buttocks, or the face has several advantages. Thus, it has become preferable to harvest intact and healthy adipocytes during a liposuction procedure.

First, however, the healthy adipocytes must be separated from damaged tissue, blood, other body fluids and any water that may have been introduced during the liposuction process. To remove these liquids, vacuum suction is applied to a lipoaspirate through a filter at the bottom of the collection flask. This vacuum suction is generally insufficient to adequately remove liquid from the lipoaspirate. Healthy adipose tissue collects on the filter, limiting the amount of liquid that may pass through it. The lipoaspirate may be agitated using a mixer such as a revolving blade. However, this is not sufficient to remove the liquid.

In view of the foregoing, there is a need to remove liquid from lipoaspirate. It is therefore desirable to provide an efficient method for removing the liquid component of a lipoaspirate.

### BRIEF SUMMARY OF THE INVENTION

Accordingly, the primary object of the present invention is to provide a method for removing liquid from a lipoaspirate.

In greater detail, aspects of the invention provide a sterile cartridge or cassette having desiccating qualities and which may be readily incorporated into a lipoaspiration collection flask.

In one embodiment, the invention provides an absorbing cartridge that may be placed in a collection flask by means of a slot in the lid.

In another embodiment, the invention provides an absorbing cartridge having a hollow body containing an absorbing material and a dessicant.

In a further embodiment, the invention provides an absorbing cartridge having a frame supporting an absorbing material.

It is therefore an object of the present invention to provide a means such as an absorption cartridge for use with a liposuction collection flask to remove a substantial portion of the liquid component of a lipoaspirate.

These and other objects and advantages of the present invention will become apparent from a reading of the attached specification and appended claims. There has thus been outlined, rather broadly, the more important features of the invention in order that the detailed description thereof that follows may be better understood, and in order that the present contribution to the art may be better appreciated. There are features of the invention that will be described hereinafter and which will form the subject matter of the claims appended hereto.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a diagram of a liposuction system of the prior art;

FIG. 2 is a

[symbol]

FIG. 3 is a side view of a collection flask in accordance with the principles of the invention;

FIG. 4 is a side view of an absorption cartridge in accordance with the principles of the invention;

FIG. 5 is a cross-sectional view of an absorption cartridge in accordance with the principles of the invention;

FIG. 6 is a side view of a collection flask and an absorption cartridge in accordance with the principles of the invention;

FIG. 7 is a side view of an alternative embodiment of a collection flask and an absorption cartridge in accordance with the principles of the invention;

FIG. 8 is a cross-sectional view of an alternative embodiment of an absorption cartridge in accordance with the principles of the invention.

### DETAILED DESCRIPTION

Before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

Disclosed is a cartridge that may be inserted into a collection flask and absorb liquids in a lipoaspirate in order to separate them from adipose tissue.

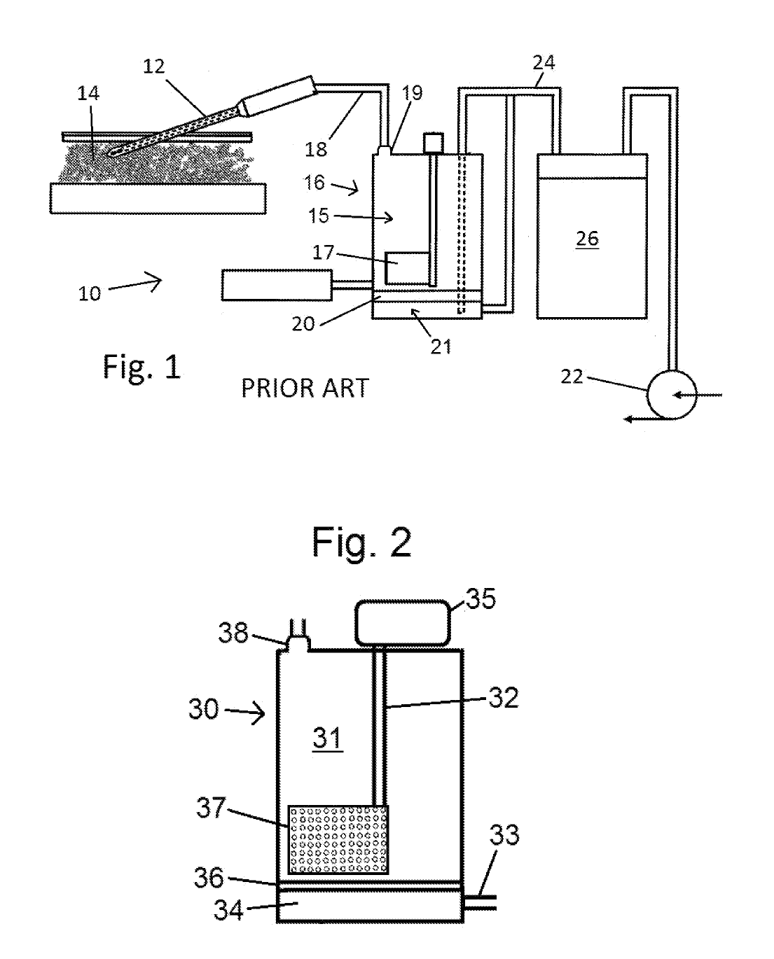

FIG. 1 shows a diagram of a typical liposuction procedure. A liposuction system 10 includes an liposuction cannula 12 that is inserted into subcutaneous fat 14. The liposuction cannula 12 may be connected to a collection flask 16 by means of a flexible tube 18. The collection flask 16 is divided into two chambers, the adipocyte collection chamber 15 and the fluid collection chamber 21 by a filter 20. The adipocyte collection chamber 15 may be connected to the flexible tube 18 at entry port 19, thereby providing fluid communication between the adipocyte collection chamber 15 and cannula 12. A vacuum pump 22 may supply vacuum pressure, i.e. negative air pressure, to the fluid collection chamber 21. This negative air pressure and gravity may both work to pull fluid out of the adipocyte collection chamber 15 and into the fluid collection chamber 21. The filter allow fluid to freely pass through it but may prevent adipocytes from passing through it. It is common for a filter 20 to be located between the entry point of tube 18 and the point where tube 24 applies suction. Typically, liquids, such as blood, water and other body fluid end up being pulled through the filter 20. A vacuum trap 26 may be placed between the fluid collection chamber 21 and the vacuum pump 22 to prevent fluids from entering and contaminating or clogging the vacuum pump 22.

When liposuction is used to harvest healthy adipocytes, relatively low pressure is used. The healthy tissue in collection flask 16 collects on top of the filter 20. Because relatively low vacuum pressure is applied, much of the liquid in collection flask 16 is not drawn through the filter 20 because it is blocked in part by the collected tissue. It is known in the art to agitate the contents of collection flask 16 by applying an agitator such as the rotating blade 17 shown in FIG. 1 or other devices, to allow more fluid to be drawn through the filter 20. However, significant agitation often results in damaging otherwise healthy adipose cells, thereby reducing the amount of adipocytes successfully harvested.

FIG. 2 shows a collection flask 30 in accordance with the principles of the invention. Collection flask 30 may be divided into two chambers by a filter 36. An adipocyte collection chamber 31 may be above the fluid collection chamber 34, with the filter 36 serving as a floor for the adipocyte collection chamber 31 and the top of the fluid collection chamber 34. A vacuum port 33 provides negative air pressure to the fluid collection chamber 34 and the entry port 38 provides fluid communication between the adipocyte collection chamber 31 and a liposuction cannula or similar device for introducing adipose tissue to the adipocyte collection chamber. The negative air pressure supplied by vacuum port 33 may result in negative pressure being imparted to the fluid collection chamber 34, which in turn provides negative air pressure through filter 36 to the adipocyte collection chamber 31 which in turn transfers negative air pressure to the entry port 38.

A source of adipose tissue may be in fluid communication with the entry port 38. A source of adipose tissue may be a liposuction cannula or previously collected adipose tissue. Adipose tissue, comprising adipocytes and lipoaspirate (which may include damaged cells, blood, water, lymph and other bodily fluids) may enter the adipocyte collection chamber 31 through entry port 38. Negative air pressure and gravity may draw fluids, but not adipocytes, through the filter 36 and into the fluid collection chamber 34. A blade 37 may rotate about axle 32 which may be powered by a small motor 35 or other means, including by hand, so that it rotates near the bottom of the adipocyte collection chamber 31 to separate the adipocytes from the lipoaspirate, thereby causing more lipoaspirate to flow downward through the filter 36.

In this embodiment, the blade 37 may also be an absorption cartridge. As with other cartridges in accordance with the principles of the invention, the blade 37 may include one or more absorptive materials, including a dessicant. Optionally the cartridge comprising the blade 37 may have negative pressure applied to its interior in order to better pull lipoaspirate out of the adipocytes the blade 37 comes into contact with. Optionally, the interior of blade 37 may be cooled to encourage fluids to condense and collect about the blade 37 or the interior of blade 37. Negative air pressure, a cooling agent and other forces may be applied to the blade cartridge 37 to facilitate removal of lipoaspirate from the adipocytes.

The absorption cartridge 37 may be disposable and comprised of a material that absorbs liquids without harming adipocytes. Optionally, the cartridge 37 may be reusable, having a frame about which absorbent material may be placed and removed after use. The cartridge 37 may optionally be a case having openings to make it permeable, or may have a web or netting about it to retain absorbent material inside it. Optionally, the cartridge 37 may be comprised of a case with nothing in it, having a liquid permeable casing and negative air pressure to suck out any liquids that enter the cartridge. Optionally, a strongly hydrophilic material may fill the cartridge and be replaced and may optionally have vacuum applied to it through the axle 37 so that no matter how much fluid is absorbed, the cartridge may still absorb more liquid.

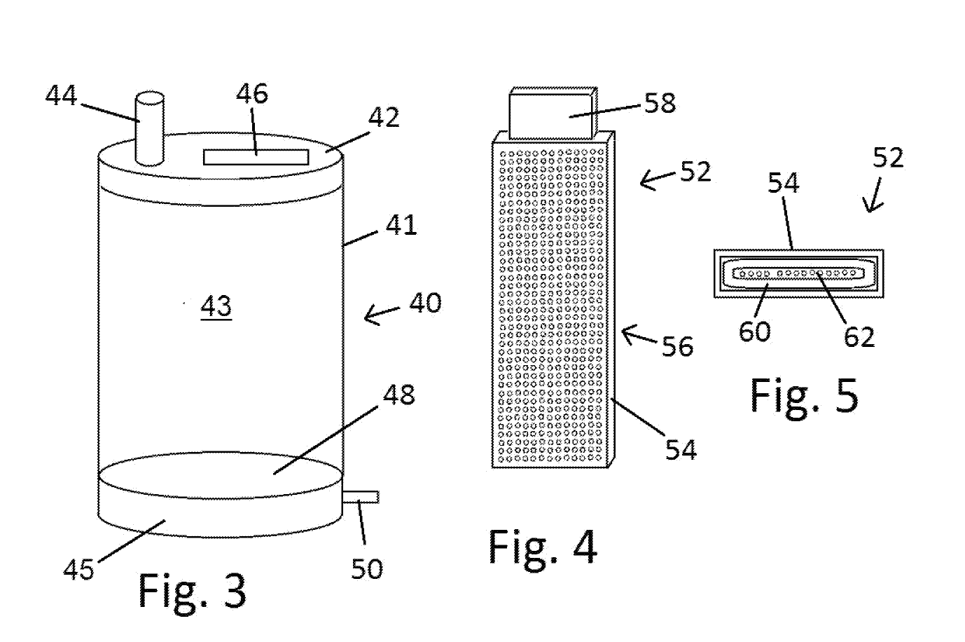

FIG. 3 shows a collection flask 40 in accordance with the principles of the present invention. Collection flask 40 includes a cylindrical side wall 41, a filter 48 dividing the collection flask 40 into an adipocyte collection chamber 43 above a fluid collection chamber 45. It may be preferable for the filter to be positioned to divide the collection flask 40 such that the adipocyte collection chamber 43 is above the fluid collection chamber 45. This may allow gravity to contribute to pulling fluid through the filter 48 and separate the adipocytes from lipoaspirate. The lid 42 may include an entry port 44 to provide fluid communication between a liposuction cannula and the adipocyte collection chamber. Optionally, the port 44 may be placed in the side wall, so long as it is on the opposite side of the filter as the vacuum port 50. The vacuum port 50 may apply negative air pressure to the fluid collection chamber 45. The lid 42 may also include a slot 46 that may optionally have an automatically closing flap or membrane at slot 46.

FIG. 4 shows an absorption cartridge 52. An absorption cartridge 52 may include a plastic parallelepiped body 54 and a handle 58. The body 54 may include a plurality of holes or perforations 56. Absorption cartridge 52 may be sized to fit within the slot 46 and may be applied therethrough to the adipocyte collection chamber 43 of the collection flask 40. The cartridge 52 may absorb fluid within the adipocyte collection chamber 43 that does not pass through the filter 48 and into the fluid collection chamber 45. Because lower negative pressure is applied during liposuction it is desired to collect healthy adipocytes, less fluid may pass through the filter 48. However, the air pressure may be reduced below ambient within the adipocyte collection chamber 43. This may allow fluid to evaporate where it may be collected by the cartridge 52. It may therefore be preferable to use a strong dessicant in the cartridge 52.

FIG. 5 shows a cross-section of the absorption cartridge 52. Within the plastic body 54 is an absorbing material 60. The absorbing material 60 may be comprised of cotton, foam, linen, and absorbent gel or the like. Any material capable of absorbing liquid may be suitable for use as absorbing material 60 in an absorption cartridge 52. In the embodiment shown in FIG. 5, a desiccant 62 is also placed within the cartridge 52. Desiccant 62 may be enclosed within an absorbing material 60 or may optionally be intermixed with it or located only within one particular region of cartridge 52. Desiccant 62 may be any material that adequately functions as a desiccant, for example, calcium chloride, calcium sulfate and the like.

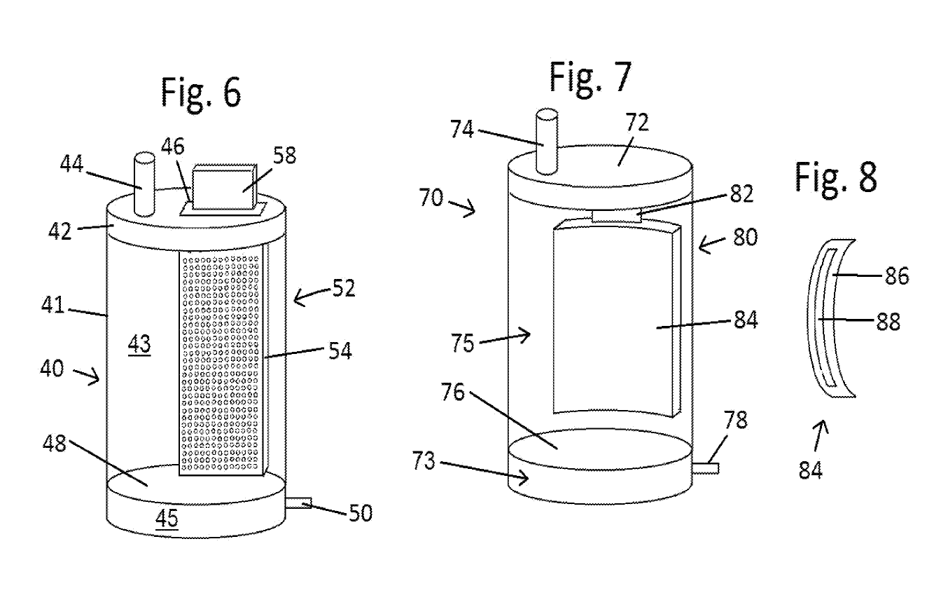

FIG. 6 shows the collection flask 40 with a cartridge 52 inserted through slot 46. Cartridge 52 may be manipulated by grasping handle 58 and sliding it into or out of slot 46 in lid 42. When cartridge 52 is exposed to the interior of flask 40, it may absorb liquids that have not been drawn through filter 48. It may be preferable for the body 54 of the cartridge 52 to be comprised of a material which adipose tissue is not likely to adhere to. The body Cartridge 52 is preferably provided in a pre-sterilized and hermetically sealed fashion. Thus, the material components of the cartridge 52, including the body 54, the absorbing material 60 and the desiccant 62 may all preferably be capable of withstanding all types of sterilization procedures. For example, a plastic and capable of withstanding the heat and pressure of a common autoclave may not be preferable in all situations in which a cartridge in accordance with the principles of the invention.

FIGS. 3-6. Show a collection flask 40 and a cartridge 52 that provides insertion and removal of a cartridge 52 through slot 46. By simply holding the cartridge 52 by its handle 58 and either pushing the cartridge 52, in or out of the slot 46. Optionally, slot 46 may be located in the wall of the flask 40 instead of in a lid. Further, the cartridge 52 may be cylindrical, or have another shape, rather than parallelepiped as shown. The cartridge 52 may also optionally be opened such that the absorbing material 60 and/or the desiccant 62 may be removed and replaced. Optionally, the cartridge 52 may be designed as a single use cartridge that is provided in a sterile package and is discarded once used. Optionally, the cartridge may be placed loosely within the collection flask.

FIG. 7 shows an alternative embodiment of an absorption cartridge in accordance with the principles of the invention. Absorption cartridge 80 may have a concave body 84 and may be affixed to the interior of the lid 72 by stem 82. Lid 72 may include an inlet nozzle 74 for connection to a liposuction cannula. Collection flask 70 may include a filter 76 as well as a nozzle 78, providing suction from the other side of the filter 76 from which lipoaspirate enters the collection flask 70. In this embodiment, the cartridge 80 may be removably affixed to the lid 72, or may be detachable from lid 72. Optionally, the cartridge 80 may be affixed, either removably or permanently, to the interior wall of collection flask 70.

FIG. 8 shows a cross-section of the concave body 84 of the absorption cartridge 80. An absorbent material 86 may be placed around a frame 88. Frame 88 may be solid or may optionally include one or more openings that may be filled with absorbent material, or a desiccant. Cartridge 80 may also be provided in an unused, sterile way and it may thus be preferable that cartridge 80 be comprised of materials that will not deteriorate when exposed to a sterilization process. In this embodiment, the absorbent material is placed about a frame rather than located inside a hollow body.

It may be desirable to optionally utilize more than one cartridge or even more than one type of cartridge in a single collection flask. Optionally, a cartridge may be designed to be pressed against collected adipose tissue within a collection flask. A cartridge and collection flask may also be designed to allow the cartridge to be manipulated and moved about while the collection flask is still sealed or even in use. It may be desirable to form the cartridge from and with materials known to not harm adipose or other tissue.

Because a variety of liquids may be found interspersed in a collection flask, it may be desirable to construct a cartridge using absorbent materials and/or desiccants which are effective in absorbing in removing both hydrophobic and hydrophilic liquids. Optionally, a cartridge may include both materials that absorb aqueous liquids and materials that absorb hydrophobic liquids.

Whereas, the present invention has been described in relation to the drawings attached hereto, it should be understood that other and further modifications, apart from those shown or suggested herein, may be made within the spirit and scope of this invention. Descriptions of the embodiments shown in the drawings should not be construed as limiting or defining the ordinary and plain meanings of the terms of the claims unless such is explicitly indicated.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

1-21. (canceled)

22. An absorption cartridge for use in a liposuction collection flask comprising an absorbing material and a frame for supporting the absorbing material, wherein the absorption cartridge is insertable into the collection flask through a slot.

23. A device for collecting healthy adipose tissue during liposuction comprising:
a collection flask;
a filter dividing the collection flask into an adipocyte collection chamber and a fluid collection chamber;
a cartridge having at least one absorptive material placed in the adipocyte collection chamber;
wherein an entry port provides fluid communication between a source of adipose tissue and the adipocyte collection chamber;
wherein a vacuum port provides negative air pressure to the fluid collection chamber;
wherein the filter allows fluid to pass through it but prevents adipocytes from passing through it.

24. The device of claim 23 further comprising a blade in the adipocyte collection chamber rotating near the filter.

25. The device of claim 23 wherein the blade is comprised of the cartridge.

26. The absorption cartridge of claim 22 wherein the frame comprises a hollow body having perforations.

27. The absorption cartridge of claim 26 wherein the slot through which the cartridge is insertable is located in a lid of the liposuction collection flask.

28. The absorption cartridge of claim 22 further comprising a desiccant within the body.

29. The absorption cartridge of claim 22 wherein the frame comprises a netting for retaining the absorbing material inside it.

30. The cartridge of claim 28 wherein negative air pressure is applied to the inside of the body.

31. The cartridge of claim 30 wherein the body is used as a blade that rotates above a filter in a liposuction collection flask;
wherein the filter separates the liposuction collection flask into an upper adipocyte collection chamber and a lower fluid collection chamber.

32. The device for collecting healthy adipose tissue during liposuction of claim 23 wherein the cartridge comprises a case having openings to make it permeable.

33. The device for collecting healthy adipose tissue during liposuction of claim 32 wherein an entry port provides fluid communication between a source of adipose tissue and the adipocyte collection chamber, a vacuum port provides negative air pressure to the fluid collection chamber and the filter allows fluid to pass through it but prevents adipocytes from passing through it.

34. The device for collecting healthy adipose tissue during liposuction of claim 32 further comprising a blade in the adipocyte collection chamber rotating near the filter.

35. The device for collecting healthy adipose tissue during liposuction of claim 32 wherein the cartridge further comprises a dessicant.

36. The device for collecting healthy adipose tissue during liposuction of claim 32 wherein the cartridge is configured to be used as a blade in the adipocyte collection chamber and rotates above the filter.

37. The device for collecting healthy adipose tissue during liposuction of claim 36 wherein negative air pressure is provided to the inside of the cartridge.

38. The device for collecting healthy adipose tissue during liposuction of claim 23 wherein the cartridge comprises a netting retaining absorbent material inside it.

39. The device for collecting healthy adipose tissue during liposuction of claim 38 wherein the cartridge further comprises a dessicant.

40. The device for collecting healthy adipose tissue during liposuction of claim 38 wherein negative air pressure is provided to the inside of the cartridge.
